(19) United States
(12) Patent Application Publication
(10) Pub. No.: US 2023/0372492 A1
(43) Pub. Date: Nov. 23, 2023

(54) DRUGS CONJUGATED WITH HEXOSE PHOSPHATE AND METHODS OF MAKING AND USING SAME

(71) Applicants: Joo Youn PARK, Starkville, MS (US); Keun Seok SEO, Starkville, MS (US); Seung Seo LEE, Southampton Hampshire (GB)

(72) Inventors: Joo Youn PARK, Starkville, MS (US); Keun Seok SEO, Starkville, MS (US); Seung Seo LEE, Southampton Hampshire (GB)

(73) Assignees: MISSISSIPPI STATE UNIVERSITY, Starkville, MS (US); UNIVERSITY OF SOUTHAMPTON, Southampton, Hampshire (GB)

(21) Appl. No.: 18/247,049

(22) PCT Filed: Sep. 29, 2021
(86) PCT No.: PCT/US2021/052550

§ 371 Date: Mar. 28, 2023

Related U.S. Application Data
(60) Provisional application No. 63/086,546, filed on Oct. 1, 2020.

(51) Int. Cl. A61K 47/54; A61P 31/04

(52) U.S. Cl. CPC A61K 47/549; A61P 31/04

(57) ABSTRACT

A drug conjugate, composition, and method for delivering active antimicrobials based on existing antibiotics through a hexose phosphate transporter (UhpT) by conjugating the antimicrobials with non-metabolizable hexose phosphates. Methods of co-administering antibiotics with non-metabolizable hexose phosphates as antimicrobials are also disclosed. Non-metabolizable hexose phosphates can constitutively and strongly induce expression of UhpT which significantly improves the efficacy and/or antimicrobial spectrum of antibiotics. This drug conjugate, composition and method will permit reuse of many FDA approved antibiotics that have been abandoned or fallen into disuse due to their current low efficacy and/or resistance to these antibiotics by pathogens.

### CROSS REFERENCE TO RELATED APPLICATIONS

This application claims the benefit of U.S. provisional application No. 63/086,546, filed Oct. 1, 2020, the entire disclosure of which is specifically incorporated herein by reference.

### FIELD OF THE INVENTION

The present disclosure relates to drug conjugates, drug compositions, methods to improve one or more of the pharmacokinetic properties of drugs such as antimicrobials by conjugating the antimicrobials with fluorinated hexose phosphates and methods to, deliver drugs such as antimicrobials using a hexose phosphate transporter by conjugating antimicrobial agents such as antibiotics with fluorinated hexose phosphates.

### BACKGROUND OF THE INVENTION

The discovery of antibiotics has saved innumerable lives over the last 75 years. However, the golden age of the antibiotic era is fading away and we are now entering a post-antibiotic dark age. Annually, in the US alone, more than 2 million hospital-acquired infections caused by multidrug resistant ESKAPE pathogens (Enterococcus faecium, Staphylococcus aureus, Klebsiella pneumoniae, Acinetobacter baumannii, Pseudomonas aeruginosa, and Enterobacter species), creating an estimated $20 billion dollars in excess healthcare costs. These ESKPE pathogens have evolved to Klebsiella pneumoniae carbapenemase (KPC)-producing bacteria, Pseudomonas aeruginosa, methicillin resistant Staphylococcus aureus (MRSA), and vancomycin resistant Enterococci (VRE) for which no treatment options remain. Despite the current antimicrobial resistance crisis, the major pharmaceutical companies are reluctant to develop new antimicrobial agents due to the average cost of about US $800 billion to develop it and the 10 years or longer time required for development. Also, pathogens soon develop resistance to new antimicrobial agents. In view of these problems, rather than developing new antimicrobial agents, the present disclosure is directed to a useful option is to reinforce currently existing antimicrobial agents by improving their efficacy and expanding their spectrum of activity.

### SUMMARY OF THE INVENTION

This disclosure provides drug conjugates, drug compositions, methods to improve one or more of the pharmacokinetic properties of drugs such as antibiotics by conjugating the antibiotics with fluorinated hexose phosphates and delivering these conjugated antibiotics to bacteria via the uptake of hexose phosphate transporter (UhpT) and methods to, deliver drugs such as antimicrobials using a hexose phosphate transporter by conjugating antimicrobial agents such as antibiotics with fluorinated hexose phosphates.

In one embodiment, this disclosure provides the mechanism by which expression of bacterial UhpT is regulated by hexose phosphates.

In another embodiment, the disclosure improves one or more of the pharmacokinetic properties of antimicrobials such as antibiotics using fluorinated hexose phosphates as carrier molecules for transporting antimicrobial agents through the UhpT.

In another embodiment, this disclosure provides a method to synthesize non-metabolizable fluorinated hexose phosphates that stably induce high levels of expression of the UhpT.

In one specific embodiment, the disclosure provides a conjugated drug including a drug conjugated to hexose phosphate or a fluorinated hexose phosphate.

The drug component of the disclosure may be a conjugate of an antimicrobial such as an antibiotic. The antibiotic may preferably be selected from, linezolid and fosfomycin.

The hexose phosphate or fluorinated hexose phosphate of the disclosure may be selected from 3-fluoro-glucose-6-phosphate, 4-fluoro-glucose-6-phosphate, 3-deoxy-3,3-difluoroglucose, 2-deoxy-2-fluoroglucose, the 2,2-difluorinated derivative of 2-deoxy-2-fluoroglucose of formula (B2):

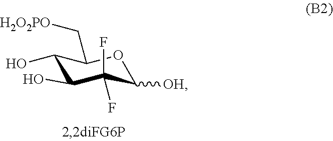

The 2,3-dideoxy-2,3-difluoroglucose, 2,3-dideoxy-2,2,3,3-tetrafluorinated analog of formula (C2):

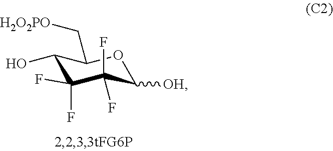

1DG6P of formula (D1)

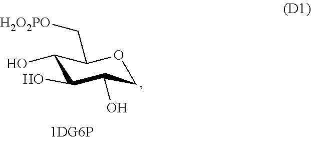

the corresponding 3-fluorinated analog of formula (D2):

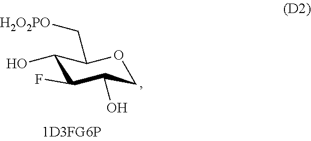

and
4-deoxy-4-fluoroglucose, and phosphate analogs of formulae (F1) and F2):

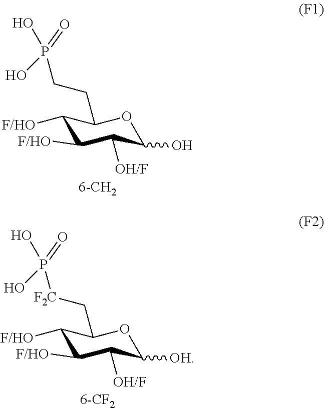

The fluorinated hexose phosphate of the present disclosure may be selected from 3-fluoro-glucose-6-phosphate or 4-fluoro-glucose-6-phosphate.

In another specific embodiment, the disclosure provides a method of using a non-metabolizable hexose phosphate that constitutively activates a HptARS regulatory system and induces expression of hexose phosphate transporter (UhpT) to modify a drug to enhance UhpT uptake of the modified drug, as compared to uptake of the unconjugated form of the same drug.

In another specific embodiment, the disclosure provides a method for conjugating a non-metabolizable hexose phosphate to a drug to enhance UhpT uptake of the conjugated drug, as compared to uptake of the unconjugated form of the same drug, said method comprising a step of reacting the drug with a non-metabolizable hexose phosphate.

The drug component of the compositions and methods of the present disclosure may be an antimicrobial and is preferably an antibiotic. Preferably, the antibiotic is selected from linezolid and fosfomycin.

The non-metabolizable hexose phosphate used in the methods of the present disclosure may be selected from 3-fluoro-glucose-6-phenylated phosphate and 4-fluoro-glucose-6-phenylated phosphate.

In another specific embodiment, the present disclosure provides a method for making 3-fluoro-glucose-6-phenylated phosphate including a step of reacting 3-fluoro-glucose with diphenyl chlorophosphate in the presence of a base.

The method for making 3-fluoro-glucose-6-phosphate may include a step of subjecting 3-fluoro-glucose to enzymatic phosphorylation to form 3-fluoro-glucose-6-phosphate.

The enzymatic phosphorylation may be carried out using hexokinase to transfer a phosphate group from adenosine triphosphate (ATP) to a 6′—OH group of 3-fluoro-glucose to form the 3-fluoro-glucose-6-phosphate.

The 3-fluoro-glucose may be formed by steps of:
(a) reacting 1,2:5,6-Di-O-isopropylidene-α-D-glucofuranose with (diethylamino)sulfur trifluoride (DAST) in a solvent to yield 3-fluoro-1,2:5,6-di-O-isopropylidene-α-D-glucofuranose, and
(b) reacting the 3-fluoro-1,2:5,6-di-O-isopropylidene-α-D-glucofuranose with trifluoroacetic acid (TFA) to remove the isopropylidene protecting group to yield the 3-fluoro-glucose.

In another embodiment, the present disclosure provides a method for making (S)—N-[[3-[3-fluoro-4-(N-1-piperazinyl)phenyl]-2-oxo-5-oxazolidinyl]methyl]acetamide comprising steps of:
(a) reacting 3,4-dinitrobenzene with piperazine to obtain 1-(2-fluoro-4-nitrophenyl)piperazine,
(b) hydrogenating the 1-(2-fluoro-4-nitrophenyl)piperazine to 1-(2-fluoro-4-aminophenyl)piperazine,
(c) protecting the amino groups of the 1-(2-fluoro-4-aminophenyl)piperazine by reaction with benzyl chloroformate to provide a protected 1-(2-fluoro-4-aminophenyl)piperazine of formula (5):

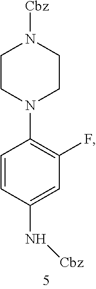 [SMILES: O=C(Nc1ccc(N2CCN(C(=O)OCc3ccccc3)CC2)c(F)c1)OCc1ccccc1]

(d) lithiating the protected 1-(2-fluoro-4-aminophenyl)piperazine of formula (5) to provide lithiated, protected 1-(2-fluoro-4-aminophenyl)piperazine,
(e) reacting the lithiated, protected 1-(2-fluoro-4-aminophenyl)piperazine with (R)-glycidyl butyrate to obtain a protected oxazolidinyl derivative of formula (7):

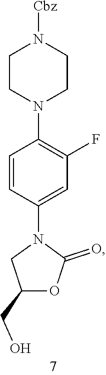 [SMILES: O=C(OCc1ccccc1)N1CCN(c2ccc(N3C[C@H](CO)OC3=O)cc2F)CC1]

(f) reacting the protected oxazolidinyl derivative of the formula (7) with tosyl chloride to provide an O-tosylated product of formula (8):

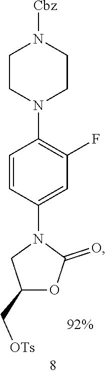 [SMILES: Cc1ccc(S(=O)(=O)OC[C@H]2CN(c3ccc(N4CCN(C(=O)OCc5ccccc5)CC4)c(F)c3)C(=O)O2)cc1]

(g) subjecting the O-tosylated product of the formula (8) to a substitution reaction with potassium phthalimide to obtain (R)—N-[[3-[3-fluoro-4-[N-1-(4-carbobenzoxy) piperazinyl]-phenyl]-2-oxo-5-oxazolidinyl]methyl]phthalimide,
(h) deprotecting the (R)—N-[[3-[3-fluoro-4-[N-1-(4-carbobenzoxy) piperazinyl]-phenyl]-2-oxo-5-oxazolidinyl]methyl]phthalimide to form a primary amine,
(i) protecting the primary amine of step (h) with an acetate to provide an N-acetyl product of formula (10):

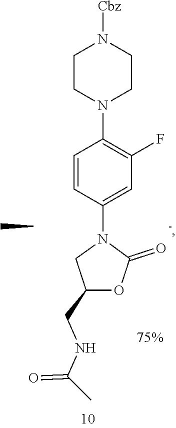

(j) deprotecting the N-acetyl product of the formula (10) by hydrogenation, optionally, using a palladium on carbon catalyst to provide (S)—N-[[3-[3-fluoro-4-(N-1-piperazinyl)phenyl]-2-oxo-5-oxazolidinyl]methyl]acetamide of formula (11):

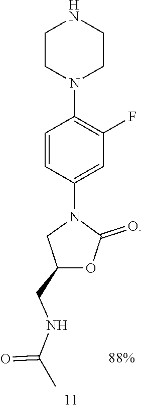 [SMILES: CC(=O)NC[C@H]1CN(c2ccc(N3CCNCC3)c(F)c2)C(=O)O1]

The disclosure also provides an example of a method of improving one or more pharmacokinetic properties of linezolid by conjugating the linezolid with 3-fluoro-glucose-6-phosphate or 4-fluoro-glucose-6-phosphate. This method may be employed to improve the antimicrobial activity of linezolid.

The disclosure also provides an example of a method of improving one or more pharmacokinetic properties of fosfomycin by conjugating it with 3-fluoro-glucose-6-phosphate or 4-fluoro-glucose-6-phosphate. This method may be employed to improve the antimicrobial activity of fosfomycin.

One method of the disclosure for conjugating 3-fluoro-glucose-6-phosphate with linezolid involves steps of:

reacting (S)—N-[[3-[3-fluoro-4-(N-1-piperazinyl)phenyl]-2-oxo-5-oxazolidinyl]methyl]acetamide of formula (11):

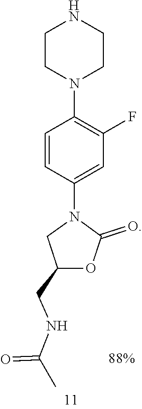 [SMILES: CC(=O)NC[C@H]1CN(c2ccc(N3CCNCC3)c(F)c2)C(=O)O1]

with γ-butyrolactone to provide an amide of the formula (14):

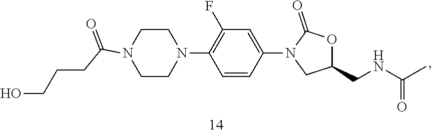 [SMILES: CC(=O)NC[C@H]1CN(c2ccc(N3CCN(C(=O)CCCO)CC3)c(F)c2)C(=O)O1]

coupling the amide of the formula (14) with a 3-fluoro-glucose-6-phenylated phosphate of formula (13):

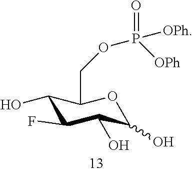

by an acid-catalyzed glycosylation reaction to produce a product of the formula (15):

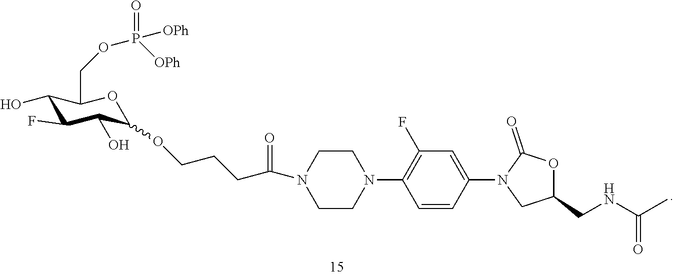

and

deprotection of the product of the formula (15) to provide a conjugated product of the formula (16):

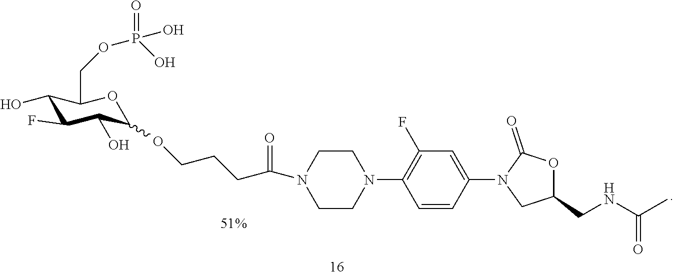

The disclosure also provides a method of treating a bacterial infection comprising administering to a patient with said bacterial infection a composition containing a conjugated antibiotic as described in any of the above embodiments.

In another specific embodiment, the disclosure relates to use of the conjugated antibiotic as described in any of the above embodiments, for treatment of a bacterial infection.

In another embodiment, the disclosure relates to a method of treating a bacterial infection comprising a step of co-administering one or more antibiotics with at least one non-metabolizable hexose phosphate or fluorinated hexose phosphate. In another embodiment, the disclosure relates to use of a non-metabolizable hexose phosphate or fluorinated hexose phosphate in combination with an antibiotic for treatment of a bacterial infection

In each of the embodiments of the previous paragraph, the hexose phosphate or the fluorinated hexose phosphate is selected from 3-fluoro-glucose-6-phosphate, 4-fluoro-glucose-6-phosphate, 3-deoxy-3,3-difluoroglucose, 2-deoxy-2-fluoroglucose, the 2,2-difluorinated derivative of 2-deoxy-2-fluoroglucose of formula (B2):

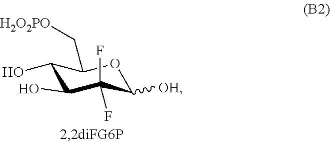

2,3-dideoxy-2,3-difluoroglucose, 2,3-dideoxy-2,2,3,3-tetrafluorinated analog of formula (C2):

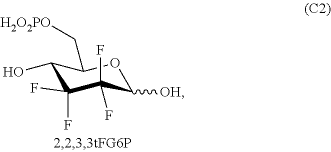

1DG6P of formula (D1):

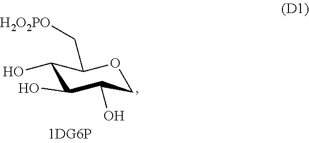

a corresponding 3-fluorinated analog of formula (D2):

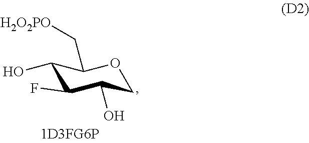

and
4-deoxy-4-fluoroglucose, and phosphate analogs of formulae (F1) and F2):

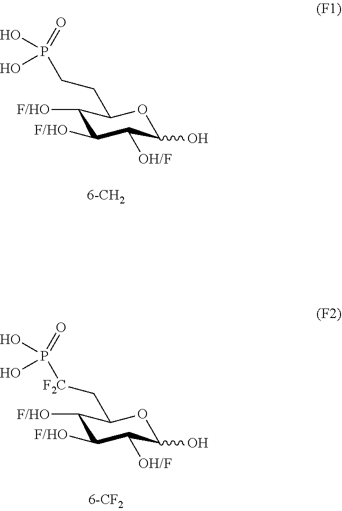

wherein F/OH indicates that the substituent can be either —F or —OH, subject to the proviso that each compound of these formulae must have at least one substituent that is —F.

While the drug conjugates, compositions, and methods of the present disclosure are illustrated and described in detail in the figures and the description herein, results in the figures and their description with linezolid or Fosfomycin conjugated with 3-fluoro-glucose-6-phosphate (3FG6P) or 4-fluoro-glucose-6-phosphate or (4FG6P) are to be considered as exemplary and not restrictive in character; it being understood that all changes and modifications that employ fluorinated hexose phosphate moeities to induce expression of UhpT and/or to facilitate transport conjugated antibiotics through UhpT are within the scope of the invention.

### BRIEF DESCRIPTION OF THE DRAWINGS

FIG. 1 illustrates the mechanism by which expression of UhpT is regulated by hexose phosphate (HP). The HptA membrane protein senses extracellular hexose phosphate which induces autophosphorylation of the HptS. Subsequently, the HptS phosphorylates the HptR which causes dimerization of the HptR. Dimerized HptR then binds to the promoter region of the uhpT gene and induces expression of UhpT to facilitate uptake of phosphates by UhpT.

FIG. 2 illustrates the use of hexose phosphate moieties to facilitate UhpT transport of antibiotics which typically could not be transported through UhpT. Hexose phosphate moieties conjugated with antibiotics (HP-AB) induce expression of UhpT which then facilitate transport of the antibiotics conjugated with hexose phosphates (AB-HP) into bacteria through UhpT due to the presence of the phosphate moiety. This provides the following effects:
1) expanding the spectrum of antimicrobial activity by transporting antimicrobials such as antibiotics lacking a suitable transport system into bacteria through UhpT, and
2) improving one or more of the pharmacokinetic properties of antimicrobials such as antibiotics by increasing expression of UhpT to thereby circumvent and potentially eliminate antimicrobial resistance.

FIG. 3 shows an example of a chemo-enzymatic method to synthesize fluorinated hexose phosphate to prevent metabolism of the synthesized hexose phosphates by hexose phosphate dehydrogenase.

FIG. 4 shows that 3-fluoro-glucose-6-phosphate (3FG6P) is not metabolized by primary canine bladder epithelial cells, 3FG6P (500 μM) was resuspended in RPMI1640 cell culture media and incubated in the presence and absence of primary canine bladder cells for a period of 24 hours. Methanol extracts of culture medium were evaporated to dryness under nitrogen gas, then reconstituted in 100 μL of 1:1 (v/v) acetonitrile/aqueous 25 mM ammonium formate. 2 μL of each sample was injected onto a 2.1 mm×100 mm HILIC column coupled to a Waters UPLC and Thermo Quantum triple-quadrupole mass spectrometer (electrospray ionization). The mass transition for 3FG6P (m/z 261.0>79.3) was monitored throughout the chromatographic run (see Figure). No evidence of metabolism of 3FG6P by bladder cells was noted.

FIG. 5 illustrates a LuxABCDE reporter system for monitoring expression of the UhpT by measuring bioluminescent light signals and strong and stable induction of UhpT expression by 3FG6P. S. aureus LAC strain harboring the LuxABCDE reporter system was cultured in a brain heart infusion broth supplemented with 500 μM glucose (Glc), glucose-6-phosphate (G6P), and 3-fluoro-glucose-6-phosphate (3FG6P) and the bioluminescent signal was monitored using a Cytation 5. While G6P temporally induced a bioluminescent signal, 3FG6P induced a constitutive bioluminescent signal. Glucose did not induce a bioluminescent signal. These results indicate that non-metabolizable 3FG6P stably activated the HptARS system and induced expression of UhpT.

FIG. 6 shows that the S. aureus LAC strain efficiently took up 3FG6P while an S. aureus LAC strain lacking UhpT (ΔUhpT) did not efficiently take up 3FG6P.

FIG. 7 shows a method to synthesize the linezolid moiety.

FIG. 8 shows a method to synthesize the 3FG6P moiety.

FIG. 9 shows a method to conjugate the linezolid moiety with 3FG6P.

FIGS. 10A to 10D show a comparison of the antimicrobial activity of linezolid and linezolid conjugated with 3FG6P against Staphylococcus aureus ATCC 25923 (FIG. 10A), Klebsiella pneumoniae ATCC 35657 (FIG. 10B), Acinetobacter baumannii ATCC BAA1605 (FIG. 10C) and Escherichia coli ATCC 25922 (FIG. 10D). The key in each of FIGS. 10A-10D applies to both graphs of each figure.

FIGS. 11A to 11C compare the antimicrobial activity of linezolid and linezolid conjugated with 3FG6P against Enterobacter cloacae ATCC 13047 (FIG. 11A), Enterobacter aerogenes ATCC13048 (FIG. 11B), and Salmonella typhimurium ATCC 14028 (FIG. 11C). The key in each of FIGS. 11A-11C applies to both graphs of each figure.

FIGS. 12A-12B show that a treatment of linezolid conjugated with 3FG6P successfully cleared a urinary tract infection caused by E. coli, while a treatment with unconjugated linezolid failed to control the infection. C57BL/6 mice were transurethrally inoculated with bioluminescent E. coli strain and treated with PBS, linezolid (80 mg/kg), and linezolid conjugated with 3FG6P (80 mg/kg) at 2, 24, and 48 hours after inoculation. 72 hours after inoculation, the progress of the infection was monitored using a IVIS Lumina XR small animal imaging system and the bacterial burden in the kidney and bladder was determined by a plate counting method.

FIGS. 13A to 13D show a comparison of the antimicrobial activity of fosfomycin alone (FIG. 13A) and fosfomycin conjugated with 3FG6P or 4FG6P (FIG. 13B) against a fosfomycin resistant E. coli clinical isolate, and fosfomycin alone (FIG. 13C) and fosfomycin conjugated with 3FG6P or 4FG6P (FIG. 13D) against an S. aureus COL strain. The key in FIG. 13D applies to all graphs of FIGS. 13A-13D.

FIGS. 14A-14B show the in vivo effect of fosfomycin or fosfomycin conjugated with 4FG6P. C57BL/6 mice (n=3) were intraperitoneally infected with bioluminescent S. aureus COL strain. After 2 and 24 hour post infection, animals were treated with fosfomycin (3 mg/kg) alone, fosfomycin conjugated with 4FG6P (50 μg/kg) or PBS as a control. After 48 hours, the progress of the infection was monitored using an IVIS Lumina XR small animal imaging system. Bacterial burdens in peritoneal lavage and tissues were determined.

### DETAILED DESCRIPTION OF THE INVENTION

Resistance to antimicrobial agents arises as a result of two main mechanisms. One mechanism involves modification of the target that the antibiotics act on by genetic mutation(s). The other mechanism prevents the antibiotic from reaching its target at a sufficiently high concentration by expressing antibiotic efflux pumps, decreasing permeability of the membrane, and/or destroying the antibiotics. The former mechanism can be addressed only by developing new antibiotics that can act on the new targets. The latter mechanism can be addressed by improving one or more of the pharmacokinetic properties of the antibiotics as in the present invention.

As used herein, the term. “hexose phosphate” may refer specifically to hexose phosphate or generically to hexose phosphate and fluorinated hexose phosphates.

As used herein, “pharmacokinetic properties” refers to one or more of drug delivery, drug absorption, drug distribution, drug metabolism, and drug excretion.

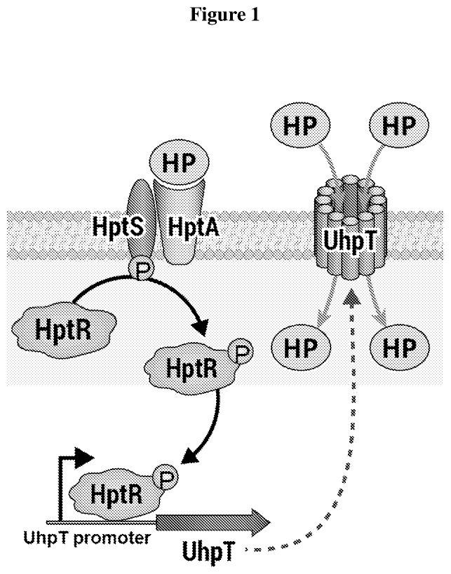

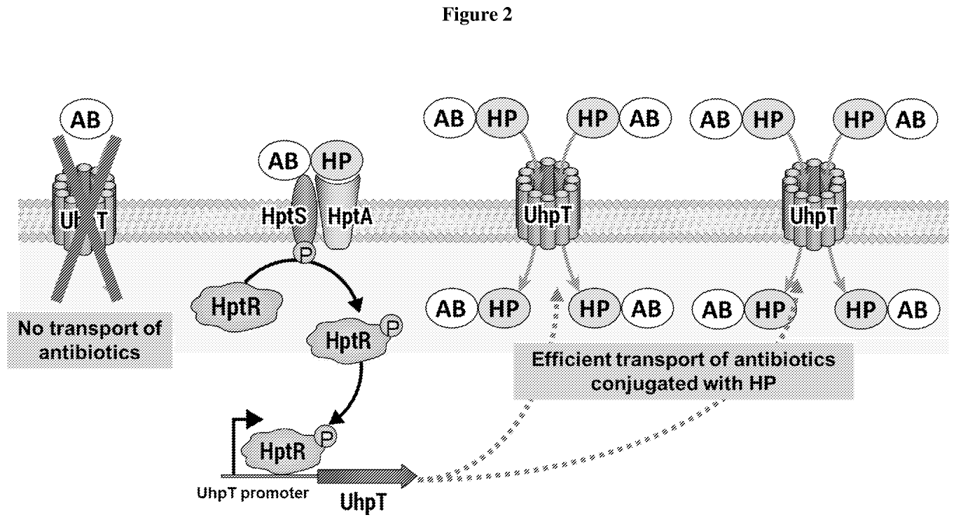

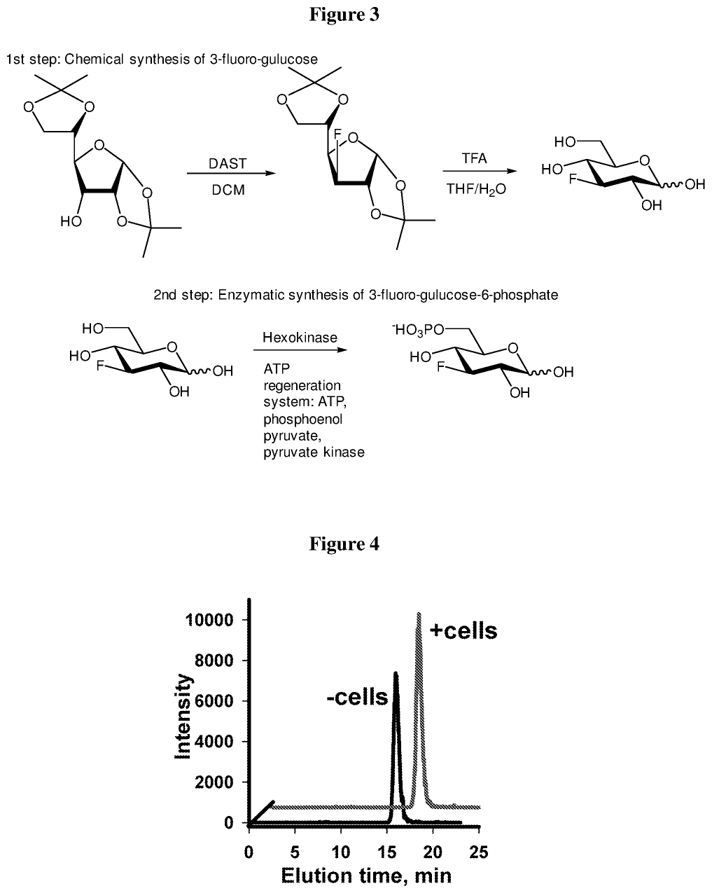

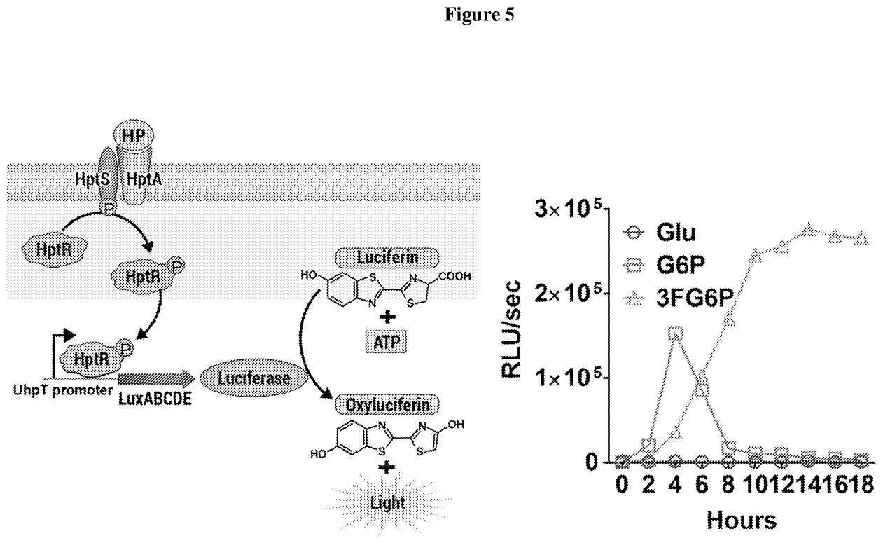

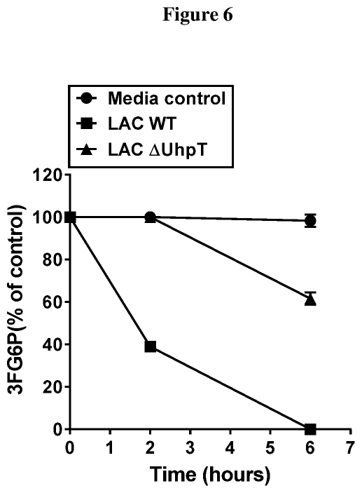

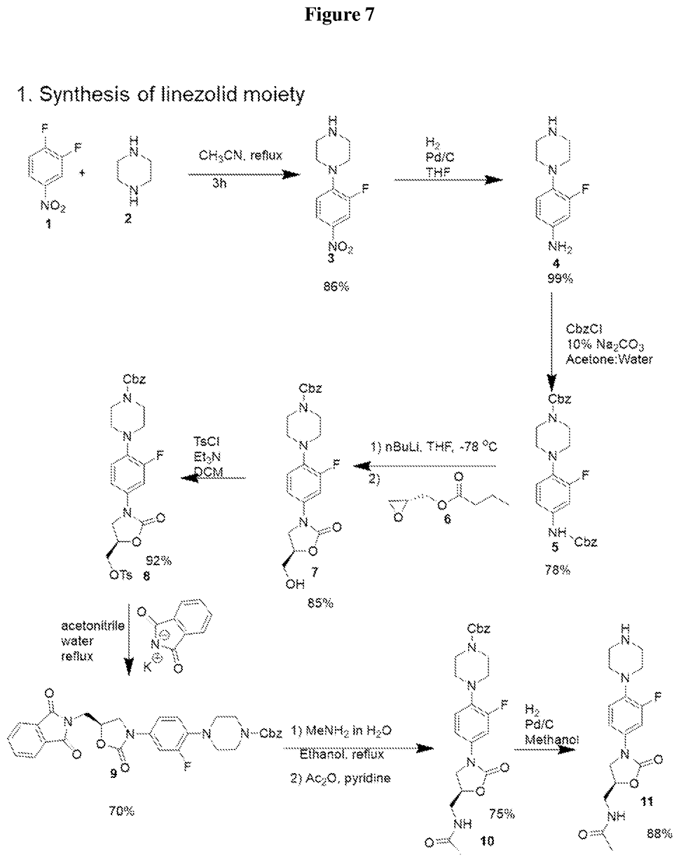

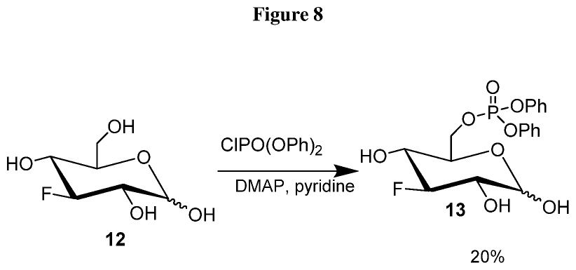

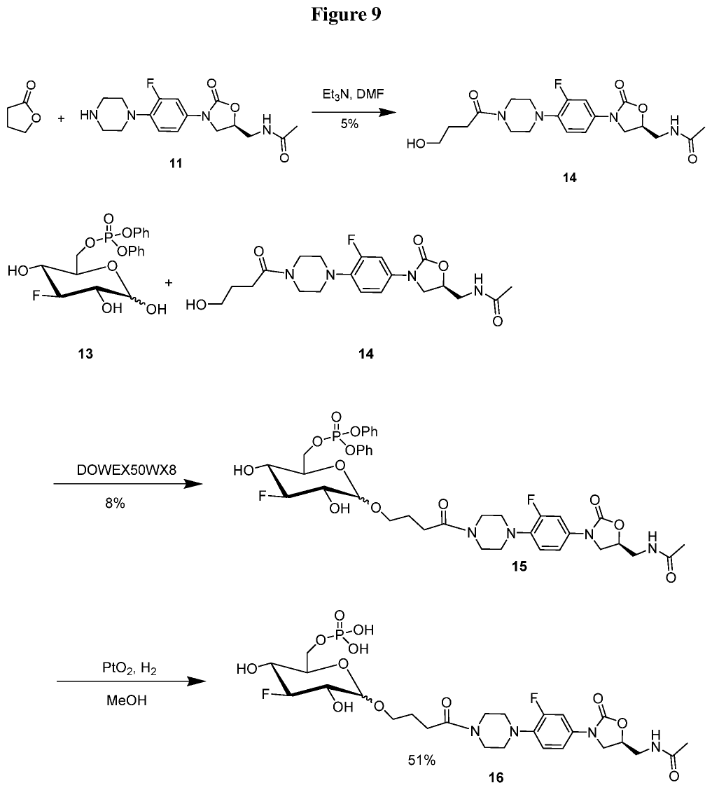

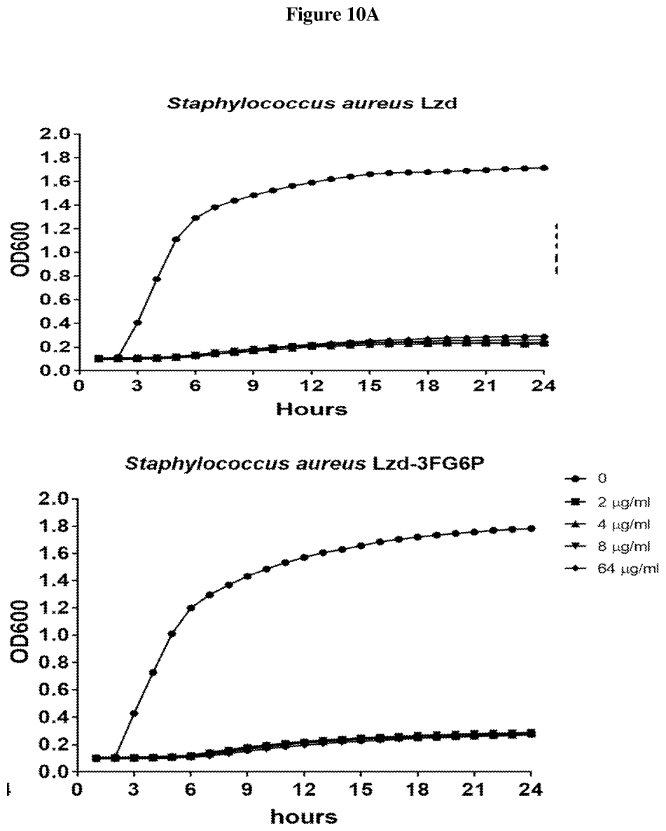

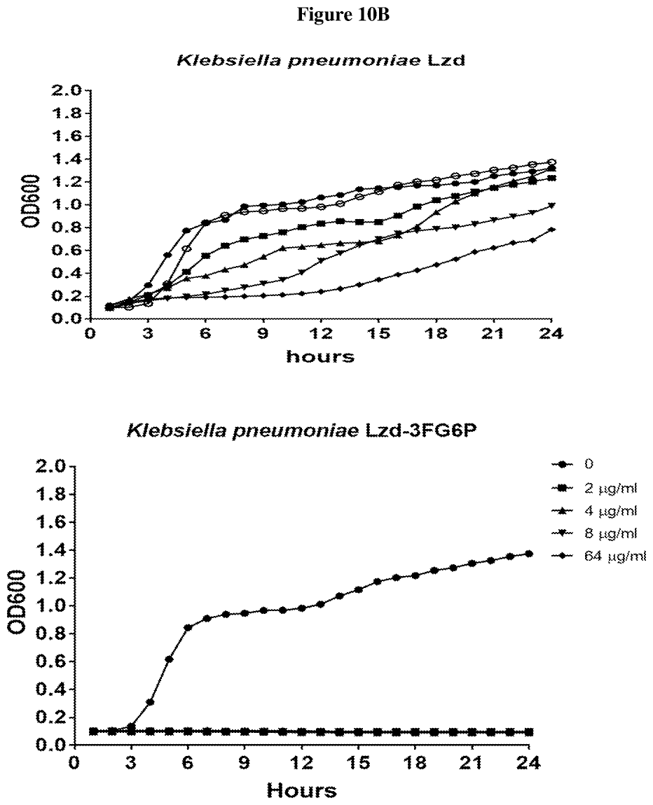

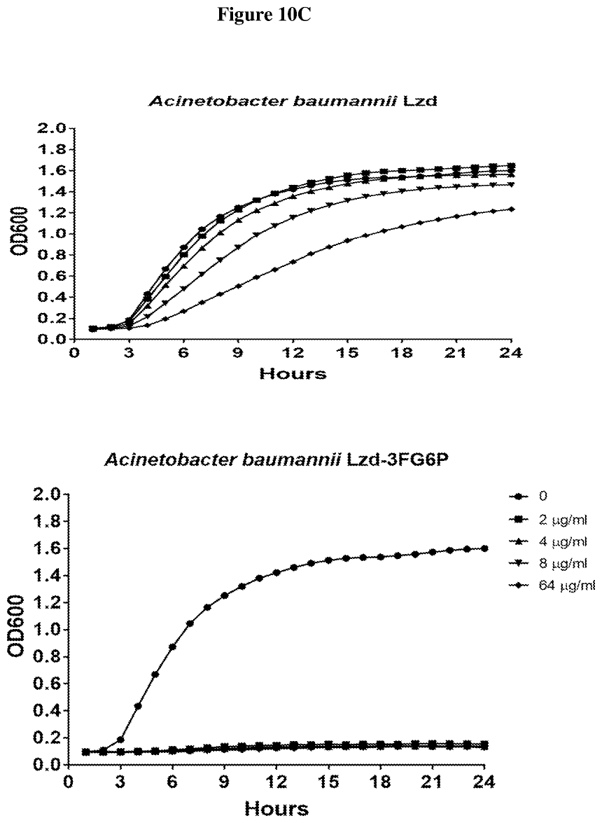

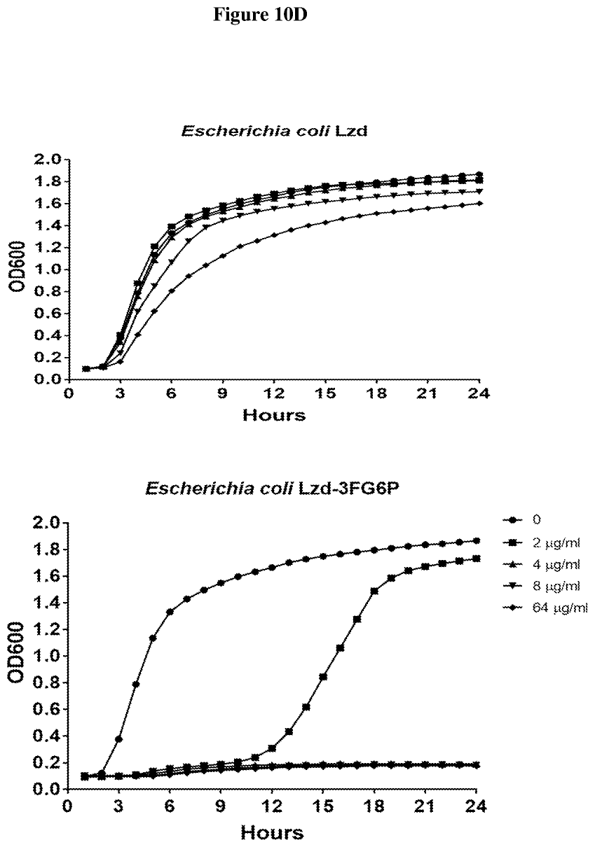

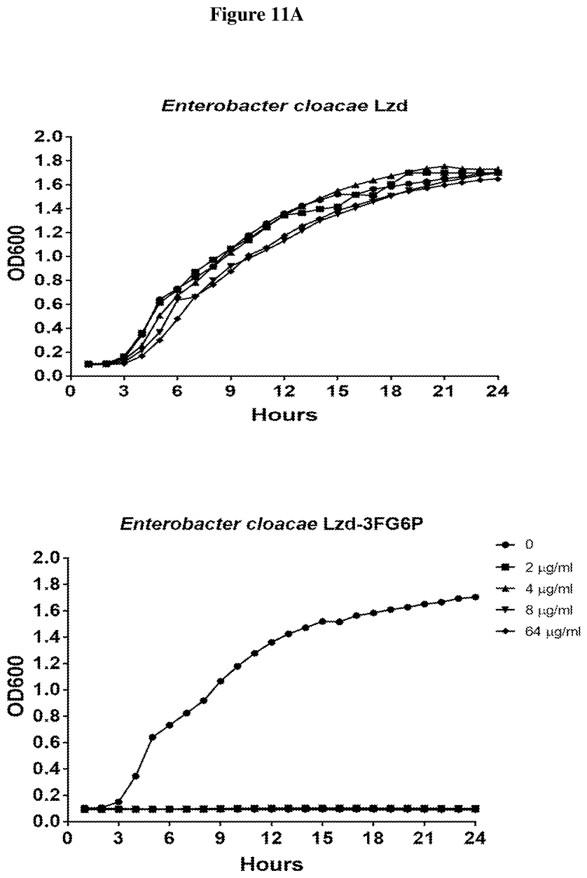

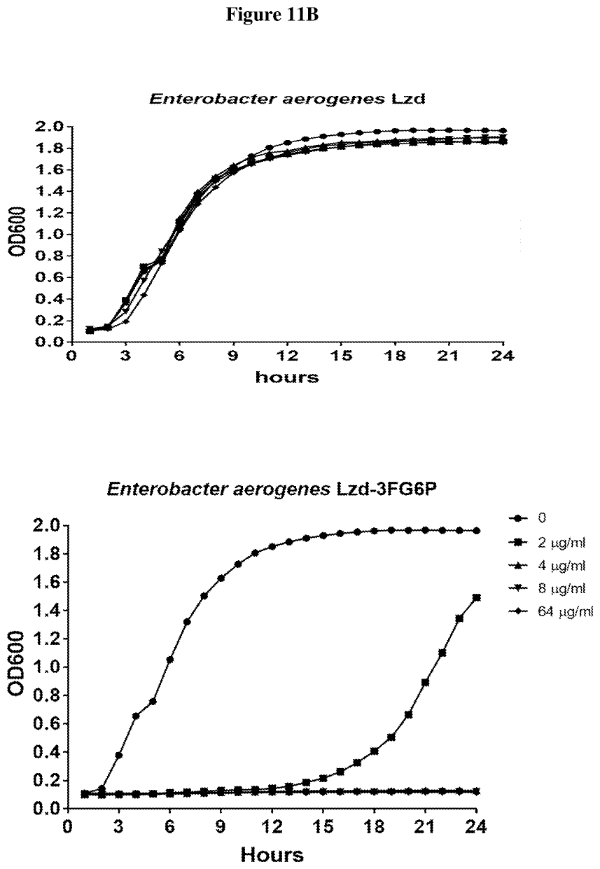

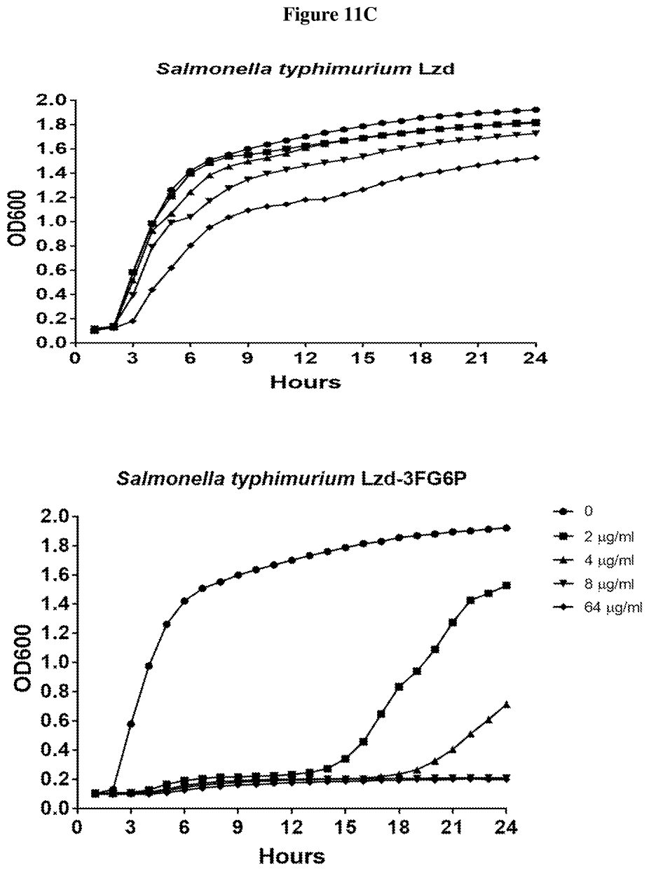

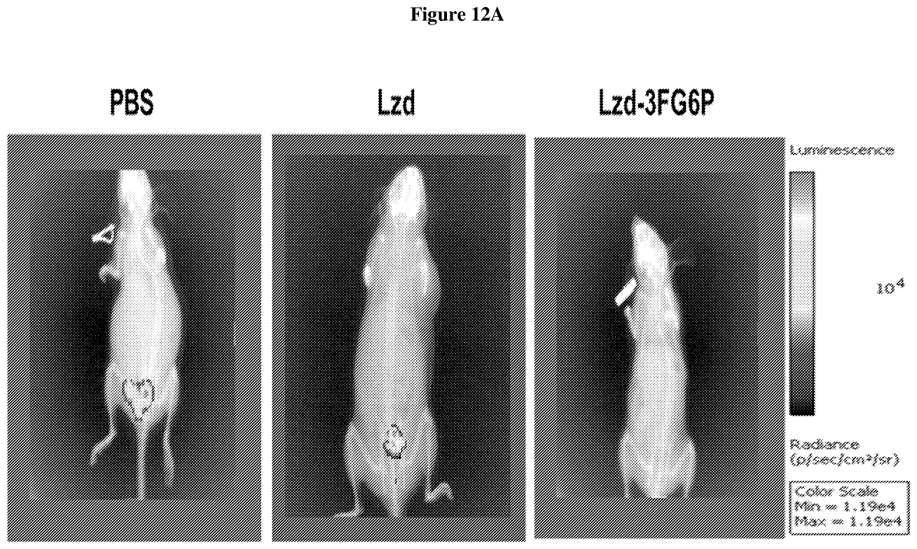

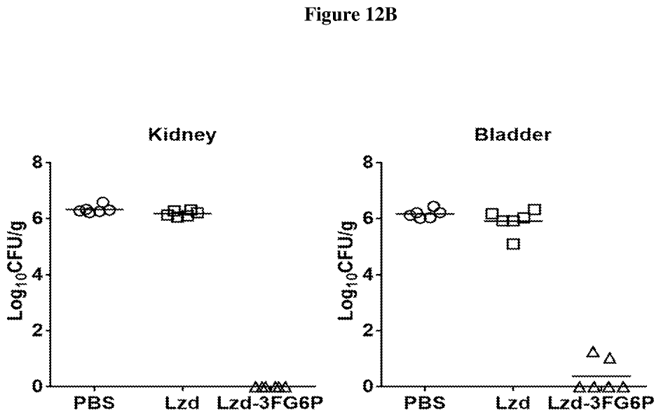

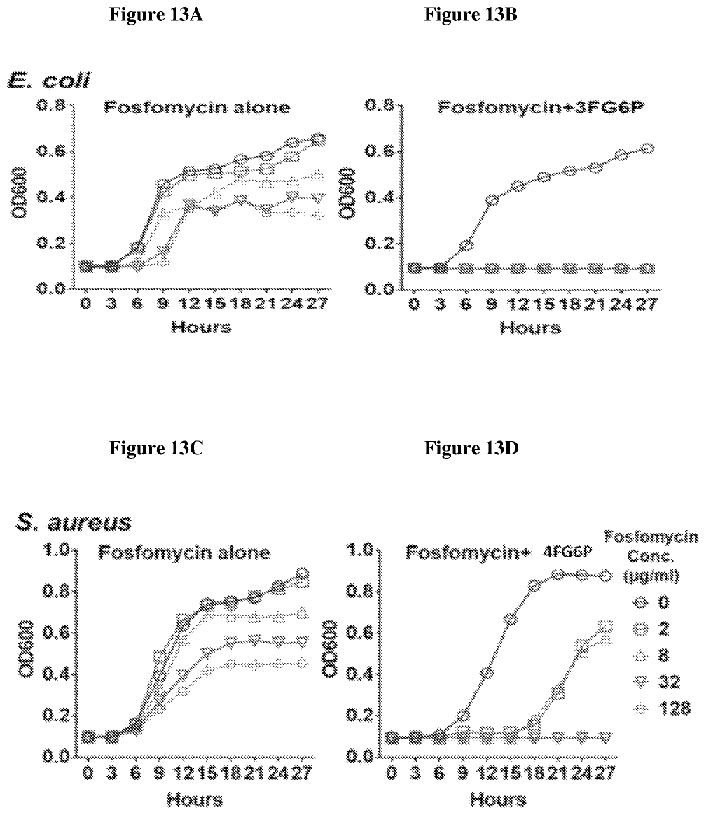

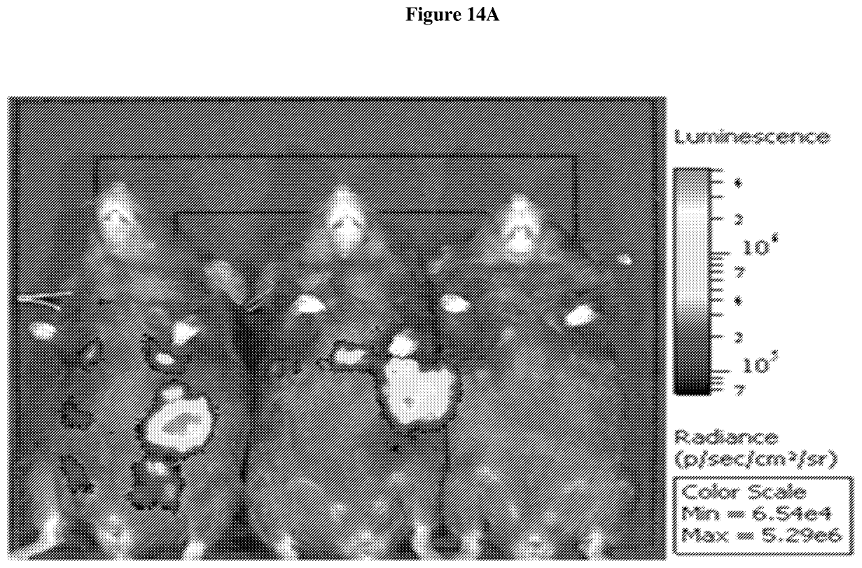

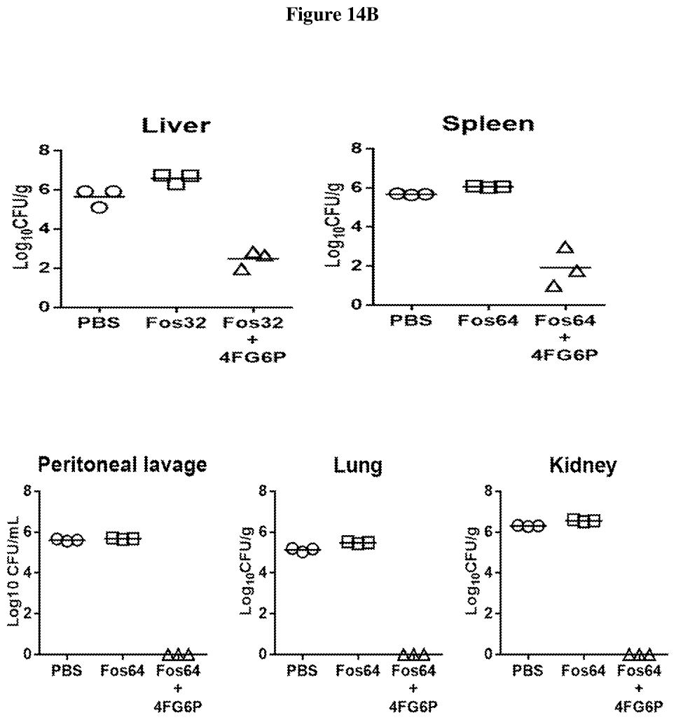

Recently, the bacterial gene regulatory system (HptARS) was characterized. HptARS controls expression of the Uptake of Hexose Phosphate Transporter (UhpT) from Staphylococcus aureus (FIG. 1). HptA is a membrane protein that senses extracellular hexose phosphate (HP). Recognition of HP by HptA induces sequential phosphorylation of HptS then HptR. HptR is a transcriptional regulator that binds to the promoter region of the UhpT gene and induces expression of UhpT to facilitate uptake of phosphorylated hexose molecules into a microbe such as bacteria.

Importantly, bacterial genome sequence analysis showed that the UhpT system is highly conserved in many gram positive and gram-negative pathogens including the ESKAPE pathogens. These findings led us to develop a concept to exploit the HptARS and UhpT system for transporting antibiotics the use of which has been discouraged due to their low efficacy and/or narrow spectrum of activity against such pathogens. More specifically, these antibiotics are conjugated with hexose phosphate (HP) or a fluorinated hexose phosphate to provide hexose phosphate conjugated antibiotics (AB-HP). The hexose phosphate conjugated antibiotics activate the HptARS system and induce expression of UhpT. This facilitates uptake of the AB-HP through UhpT. Unconjugated antibiotics (AB) lacking the hexose phosphate moiety could not be transported through UhpT or otherwise into bacteria due to the lack of a suitable transport system. This conjugation with hexose phosphate or a fluorinated hexose phosphate increases the efficacy of such antibiotics and expands their spectrum of antimicrobial activity. This conjugation with hexose phosphate or a fluorinated hexose phosphate is applicable to many antibiotics that have been abandoned or discouraged for use due to their current low efficacy and/or narrow spectrum of antimicrobial activity.

The present disclosure relates to the conjugation of an antimicrobial such as an antibiotic to a hexose phosphate moiety via a linker. The linker may be selected to be relatively easy to conjugate to drugs to ensure versatile use with a diverse array of drugs such as antibiotic molecules. The linker may be a cleavable linker or a non-cleavable linker. Preferably, the linker is a cleavable linker since, in many cases, the antibiotic molecule needs to be released once inside the bacteria to provide the desired effect. However, depending on the SAR of the antibiotic molecule, a non-cleavable linker could be used in order to offer an enhanced stability.

Table 1 below shows a list of exemplary suitable antibiotics and, cleavable linkers of the present disclosure.

TABLE 1
A list of small molecule antibiotics and linkers

| Linkers | Drugs |
|---|---|
| 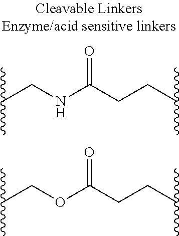 [SMILES: *CCC(=O)NC* ; *CCC(=O)OC*] | 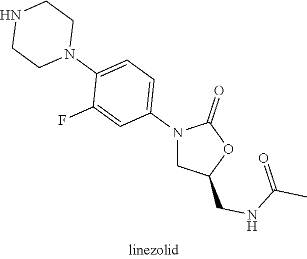 [SMILES: CC(=O)NC[C@H]1CN(c2ccc(N3CCNCC3)c(F)c2)C(=O)O1] |
| 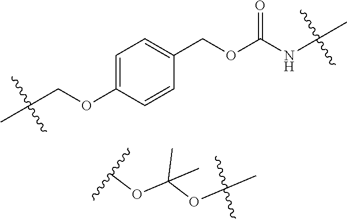 | 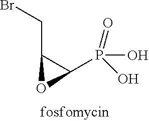 |
| | 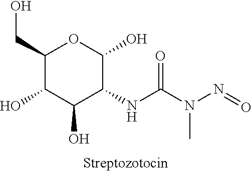 [SMILES: CN(N=O)C(=O)N[C@@H]1[C@@H](O)[C@H](O)[C@@H](CO)O[C@@H]1O] |
| 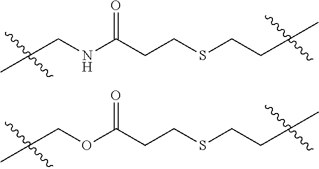 [SMILES: *CCSCCC(=O)NC* ; *CCSCCC(=O)OC*] | 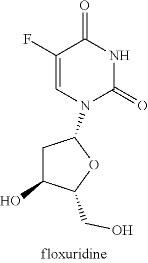 [SMILES: O=c1[nH]c(=O)n([C@H]2C[C@H](O)[C@@H](CO)O2)cc1F] |
| 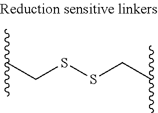 [SMILES: *CSSC*] | |
| 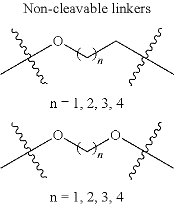 | 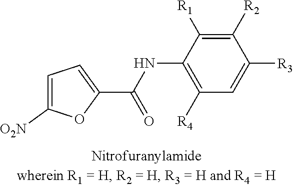 |
| | 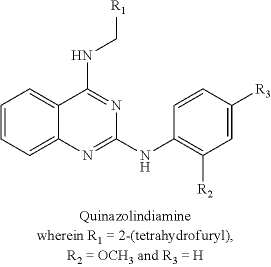 |
| | 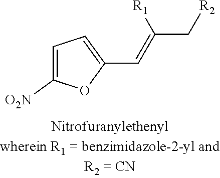 |
| | 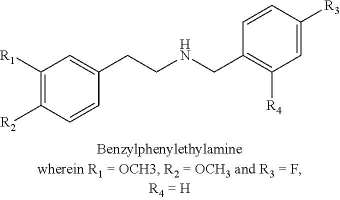 |
| | 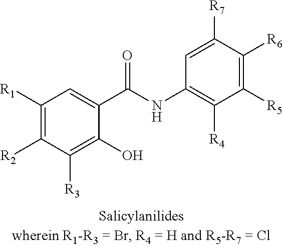 |

The third element of the conjugated drug is a glucose-6-phosphate (G6P) unit that is preferably stabilized against being metabolized. 3FG6P is one example of a suitable moiety that is sufficiently active as well as resistant to metabolism. 4FG6P and other FG6Ps may also be used. Suitable fluorinated hexose phosphates also include 3-deoxy-3,3-difluoroglucose (A), 2-deoxy-2-fluoroglucose (B1), the 2,2-difluorinated derivative of 2-deoxy-2-fluoroglucose (B2), the 2,3-dideoxy-2,3-difluoroglucose (C1) and 2,3-dideoxy-2,2,3,3-tetrafluorinated analogs (C2). Deoxygenation at the 1-position would prevent both the glycolysis and pentose phosphate pathways, thus 1DG6P (D1) and the corresponding 3-fluorinated analogs (D2) may also be suitable, as is 4-deoxy-4-fluoroglucose (E).

Suitable methods for the synthesis of the polyfluorinated compounds are available in the literature. Hexokinase can phosphorylate mono-fluorinated glucoses. For substrates that cannot be converted by hexokinase, chemical phosphorylation can be used.

A second type of suitable analogs includes the 6-phosphate group (F). Fluorination in the sugar ring does not affect the third avenue of metabolism, hydrolysis of a phosphate group. This can be addressed via the synthesis of non-hydrolysable phosphate analogs by replacement of the bridging oxygen of the phosphate with either methylene (F1) or a fluorinated methylene group (F2).

Where a substituent is indicated as “F/OH” in the formulae below, this indicates that the substituent can be either —F or —OH, subject to the proviso that each compound of these formulae must have at least one substituent that is —F.

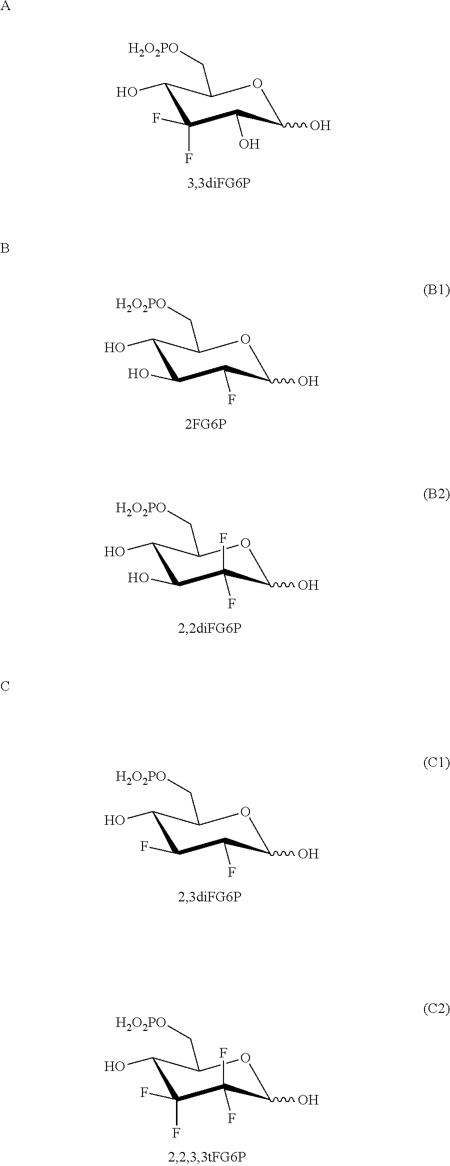

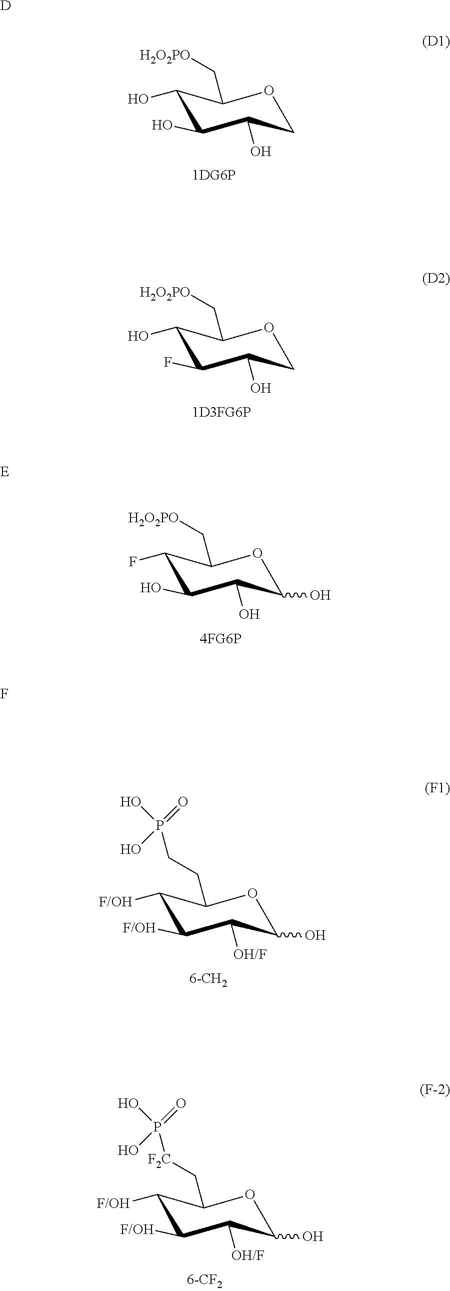

Hexose phosphates are highly metabolizable nutrients that provide energy to bacteria and host cells. Therefore, normal hexose phosphates would have a very short half-life. To improve the pharmacodynamics of hexose phosphates, it is necessary to develop hexose phosphates that are not readily metabolized by bacteria and host cells. Fluorination modulates the electronic properties of the molecule, and it is known that fluorination of ligands allows attractive interactions with protein residues which can, in most cases, favorably modulate the binding affinity of carbohydrates to proteins. A recent study demonstrated that fluorinated carbohydrates can provide protection from enzymatic degradation in Mycobacterium. See Marriner G A, Kiesewetter D O, D'Hooge F, Lee S S, Boutureira O, Raj R, Khan N, Via L E, Barry C E, Davis B G. Evaluation of Trehalose Derivatives as Radiotracers Specific for Tuberculosis in Animal Models of Disease. Journal of Labelled Compounds and Radiopharmaceuticals. 2015; 58(S1):S250. doi: 10.1002/jlcr.3302_2.

In other aspects, the disclosure provides methods to improve one or more of the pharmacokinetic properties of drugs such as antibiotics by conjugating the antibiotics with fluorinated hexose phosphates and delivering these conjugated antibiotics to bacteria via the uptake of hexose phosphate transporter (UhpT) and methods to, deliver drugs such as antimicrobials using a hexose phosphate transporter by conjugating antimicrobial agents such as antibiotics with hexose phosphate or fluorinated hexose phosphates. Examples of methods of improving one or more pharmacokinetic properties of linezolid and fosfomycin by conjugating the linezolid or fosfomycin with 3-fluoro-glucose-6-phosphate or 4-fluoro-glucose-6-phosphate are described above. These methods may be employed to improve the antimicrobial activity of linezolid and fosfomycin.

This disclosure also provides the mechanism by which expression of bacterial UhpT is regulated by hexose phosphates.

In another embodiment, this disclosure provides a method to synthesize non-metabolizable fluorinated hexose phosphates that stably induce high levels of expression of the UhpT.

In another specific embodiment, the present disclosure provides a method for making 3-fluoro-glucose-6-phenylated phosphate including a step of reacting 3-fluoro-glucose with diphenyl chlorophosphate in the presence of a base as described in greater detail above.

The disclosure also provides a method of treating a bacterial infection comprising administering to a patient with said bacterial infection a composition containing a conjugated antibiotic as described in any of the above embodiments.

In another specific embodiment, the disclosure relates to use of the conjugated antibiotic as described in any of the above embodiments, for treatment of a bacterial infection.

In another embodiment, the disclosure relates to a method of treating a bacterial infection comprising a step of co-administering one or more antibiotics with at least one non-metabolizable hexose phosphate or fluorinated hexose phosphate. In another embodiment, the disclosure relates to use of a non-metabolizable hexose phosphate or fluorinated hexose phosphate in combination with an antibiotic for treatment of a bacterial infection

In each of the embodiments of the previous paragraph, the hexose phosphate or the fluorinated hexose phosphate is selected from 3-fluoro-glucose-6-phosphate, 4-fluoro-glucose-6-phosphate, 3-deoxy-3,3-difluoroglucose, 2-deoxy-2-fluoroglucose, the 2,2-difluorinated derivative of 2-deoxy-2-fluoroglucose of formula (B2):

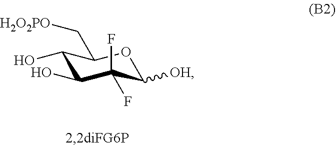

2,3-dideoxy-2,3-difluoroglucose, 2,3-dideoxy-2,2,3,3-tetrafluorinated analog of formula (C2):

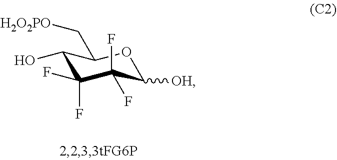

1DG6P of formula (D):

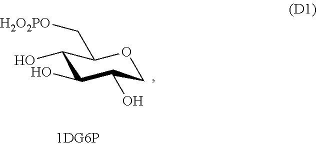

a corresponding 3-fluorinated analog of formula (D2):

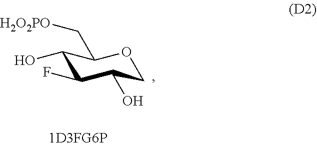

and 4-deoxy-4-fluoroglucose, and phosphate analogs of formulae (F1) and F2):

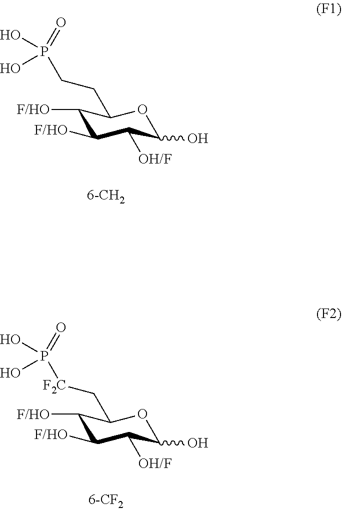

wherein F/OH indicates that the substituent can be either —F or —OH, subject to the proviso that each compound of these formulae must have at least one substituent that is —F.

### EXAMPLES

### Example 1: Synthesis of 3-fluoro-glucose-6-phosphate

In a first step, 3-fluoro-glucose-6-phosphate is synthesized whereby a hydroxyl group at the third carbon of the glucose ring is replaced by a fluorine atom. The first step in this synthesis is the making of 3-fluoro-glucose. In this step, 1,2:5,6-Di-O-isopropylidene-α-D-glucofuranose was reacted with (diethylamino)sulfur trifluoride (DAST) in dichloromethane, which resulted in stereospecific fluorination at the 3′-position, yielding 3-fluoro-1,2:5,6-Di-O-isopropylidene-α-D-glucofuranose. The 3-Fluoro-1,2:5,6-di-O-isopropylidene-α-D-glucofuranose was then reacted with trifluoroacetic acid (TFA) to remove the isopropylidene protecting group, which yielded 3-fluoro-glucose. 3-fluoro-glucose was purified by flash chromatography and subjected to enzymatic phosphorylation to form 3-fluoro-glucose-6-phosphate. In this reaction, hexokinase transfers a phosphate group from ATP to the 6′—OH group of 3-fluoro-glucose (FIG. 3). The product, 3-fluoro-glucose-6-phosphate was then purified by HPLC equipped with an anion exchange column and lyophilized until use.

To test whether 3-fluoro-glucose-6-phosphate (3FG6P) is metabolized by host cells, 3FG6P (500 μM) was resuspended in RPMI1640 cell culture media and incubated in the presence and absence of primary canine bladder cells for a period of 24 h. Methanol extracts of culture medium containing x were evaporated to dryness under nitrogen gas, then reconstituted in 100 μL of 1:1 (v/v) acetonitrile/aqueous 25 mM ammonium formate. 2 μL of each sample was injected onto a 2.1 mm×100 mm HILIC column coupled to a Waters UPLC and Thermo Quantum triple-quadrupole mass spectrometer (electrospray ionization). The mass transition for 3FG6P (m/z 261.0>79.3) was monitored throughout the chromatographic run. The intensity of 3FG6P peak from cell culture media in the presence of canine bladder epithelial cells was not different from the peak for the same cell culture media in the absence of canine bladder epithelial cells indicating that 3FG6P is not metabolized by bladder epithelial cells (FIG. 4).

### Example 2: Effect of 3-fluoro-glucose-6-phosphate on UhpT

To test whether 3FG6P can induce expression of the UhpT, we generated a bioluminescent reporter plasmid in which the promoter region of the uhpT was fused to the promoterless LuxABCDE operon (FIG. 5). See Francis K P, Joh D, Bellinger-Kawahara C, Hawkinson M J, Purchio T F, Contag P R. Monitoring bioluminescent Staphylococcus aureus infections in living mice using a novel luxABCDE construct. Infection and immunity. 2000; 68(6):3594-600. Epub 2000/05/19. PubMed PMID: 10816517; PMCID: PMC97648 and Karsi A, Lawrence M L. Broad host range fluorescence and bioluminescence expression vectors for Gram-negative bacteria. Plasmid. 2007; 57(3):286-95. Epub 2007/01/09. doi: 10.1016/j.plasmid.2006.11.002. PubMed PMID: 17207855 for monitoring of the expression of UhpT by measuring bioluminescent light signals. While glucose-6-phosphate temporally induced bioluminescent light signals for 8 hours, 3FG6P induce significantly higher bioluminescent light signals for more than 18 hours (FIG. 5). As expected, glucose did not induce any bioluminescent light signals. These results indicate that 3FG6P stably and strongly activated the HptARS system and induced expression of UhpT.

To use 3FG6P as a carrier molecule to transport antibiotics into bacteria, the 3FG6P has to be recognized by UhpT even after conjugation to the antibiotic. To test this, we generated Staphylococcus aureus LAC lacking the uhpT gene (ΔUhpT), which, as a result, is unable to transport hexose phosphates. The PBS containing 3FG6P (500 PM) was incubated in the presence of both S. aureus LAC wild-type (WT) strain and the S. aureus LAC ΔUhpT strain, and in the absence of S. aureus (PBS control) for 6 hours. The concentration of 3FG6P was determined using an HILIC column coupled to a Waters UPLC and Thermo Quantum triple-quadrupole mass spectrometer as described above. When incubated with S. aureus LAC wild type (WT) strain for 2 hours, the 3FG6P concentration rapidly decreased to approximately 40% to the PBS control, and completely disappeared from PBS in 6 hours (FIG. 6). By contrast, when incubated with S. aureus LAC ΔUhpT strain, 100% of the 3FG6P remained for 2 hours and then the concentration of the 3FG6P gradually decreased to approximately 60% to the PBS control in 6 hours. These results demonstrate that 3FG6P is efficiently transported into S. aureus WT strain by UhpT.

Collectively, these results demonstrate that fluorinated hexose phosphate (3FG6P) is able to induce stable and strong expression of UhpT and, despite the fluorine modification, fluorinated hexose phosphate (3FG6P) is still effectively recognized and transported into bacterial cells by UhpT. These results prove the concept that fluorinated hexose phosphates can be used as carrier molecules to transport antibiotics into bacterial cells via UhpT. This will enable reuse of antibiotics that have fallen out of favor due to their current low efficacy and/or narrow spectrum of antimicrobial activity.

### Example 3: Expanding Antimicrobial Activity of Linezolid by Conjugating with 3FG6P

Linezolid is a member of the family of 3-aryl-2-oxazolidinones which have an acetamidomethyl group attached to the 5-position of the oxazolidinone ring and fluorine substitutions at the 3 position of the phenyl group. As used herein, linezolid refers to (S)—N-[[3-[3-fluoro-4-(N-1-piperazinyl)phenyl]-2-oxo-5-oxazolidinyl]methyl]acetamide. Linezolid inhibits bacterial ribosomal protein synthesis at a very early stage. Linezolid binds to the 23S of the 50S ribosomal subunit which prevents the formation of a functional 70S initiation complex with the 30S subunit, fMet-RNA, initiation factors IF2 and IF3, and mRNA.

Linezolid is effective against all clinically important Gram-positive bacteria such as methicillin-resistant Staphylococcus aureus (MRSA) with MIC90s ranging from 1-4 and 2 μg/ml, Staphylococcus epidermidis (MRSE) with MIC90s of 1-4 and 1 μg/ml, vancomycin-resistant Enterococcus (VRE)faecalis and faecium with MIC90s of 1-4 and 2 μg/ml. However, linezolid is less effective against aerobic Gram-negative pathogens due to their rapid efflux mechanisms. Linezolid is not active against Acinetobacter spp, Escherichia coli, Klebsiella pneumoniae. Proteus penneri, Pseudomonas aeruginosa, and Stenotrophomonas maltophilia. Linezolid displays minimal activity against Haemophilus influenzae and Neisseria gonorrhea, with MIC90s of 16 μg/ml. See Jones R N. Johnson D M, Erwin M E. In vitro antimicrobial activities and spectra of U-100592 and U-100766, two novel fluorinated oxazolidinones. Antimicrob Agents Chemother 1996; 40(3):720-726 and Zhanel G G, Karlowsky J A, Low D E. Hoban D J. Antibiotic resistance in respiratory tract isolates of Haemophilus influenzae and Moraxella catarrhalis collected from across Canada in 1997-1998. J Antimicrob Chemother 2000; 45(5):655-66.

Synthesis of Linezolid Conjugated with 3FG6P

To demonstrate that fluorinated hexose phosphates can improve the efficacy and/or the spectrum of antimicrobial activity of linezolid, linezolid was conjugated with 3FG6P. The synthesis of linezolid conjugated with 3FG6P (17) was carried out in three parts, firstly by synthesis of linezolid moiety (S)—N-[[3-[3-fluoro-4-(N-1-piperazinyl)phenyl]-2-oxo-5-oxazolidinyl]methyl]acetamide (11) from 3,4-dinitrobenzene (1) in a series of 8 steps. 3,4-dinitrobenzen (1) was first reacted with piperazine to obtain 1-(2-fluoro-4-nitrophenyl)piperazine, which was subsequently reacted to give N-protected derivative (5). N-protected derivative (5) was then lithiated using n-BuLi and subsequently reacted with (R)-glycidyl butyrate (6) to obtain an oxazolidinyl derivative (7) which was then reacted with tosyl chloride to provide an O-tosylated product (8). The O-tosylated product (8) was made to undergo substitution reaction with potassium phthalimide to obtain (R)—N-[[3-[3-fluoro-4-[N-1-(4-carbobenzoxy) piperazinyl]-phenyl]-2-oxo-5-oxazolidinyl]methyl]phthalimide (9). Deprotection of (9) to primary amine and further protection with an acetate gave the N-acetyl product (10) which was deprotected with H2 and palladium on carbon to give the desired product (11) shown in FIG. 7.

The synthesis of 3-fluoro-glucose-6-phenylated phosphate moiety (13) was carried out by reacting 3-fluoro-glucose with diphenyl chlorophosphate in the presence of the base, 4-dimethylamino pyridine (DMAP) (FIG. 8).

The product (11) was then reacted with γ-butyrolactone to the intermediate amide (14), which was subsequently coupled with the 3-fluoro-glucose-6-phenylated phosphate moiety (13) via an acid-catalyzed glycosylation reaction to afford product (15). Final deprotection yields the desired product (16) (FIG. 9).

### Example 4: In Vitro Antimicrobial Activity of Linezolid and Linezolid Conjugated with 3FG6P

To test the antimicrobial activity of linezolid and linezolid conjugated with 3FG6P (Lzd-3FG6P) against Gram-positive and Gram-negative pathogens, the pathogens Staphylococcus aureus ATCC 25923, Klebsiella pneumoniae ATCC 35657, Acinetobacter baumannii ATCC BAA1605, Escherichia coli ATCC 25922, Enterobacter cloacae ATCC 13047, Enterobacter aerogenes ATCC13048, and Salmonella typhimurium ATCC 14028 were used. The minimal inhibitory concentration (MIC) was determined using broth microdilution following the instructions of Clinical and Laboratory Standards Institute (CLSI) document M07-A9 (Clinical and Laboratory Standards Institute. 2012. M07-A9. Methods for dilution antimicrobial susceptibility tests for bacteria that grow aerobically: approved standard. 9th ed. Clinical and Laboratory Standards Institute, Wayne, PA). Briefly, these bacterial strains were grown in Muller Hinton broth (MHB) until exponential phage (OD600<1.0). The exponentially grown testing bacteria were diluted to an OD600 of 0.01 in MHB and aliquoted in a 96 well plate. Stocks of linezolid (Lzd) and linezolid conjugated with 3FG6P (Lzd-3FG6P) were prepared at 10 mg/ml in DMSO which was diluted by two-fold serial dilutions from 64 μg/ml to 2 μg/ml (final concentration) and added to the 96 well plate. A DMSO control (0 μg/ml in the figures) was included. The plate was incubated at 37° C. and the growth of bacteria was monitored by measuring OD600 using a Cytation 5 plate reader (BioTek).

While linezolid successfully inhibited the growth of the Gram-positive pathogen, Staphylococcus aureus ATCC 25923 with a MIC of less than 2 μg/ml, linezolid failed to inhibit the growth of Gram-negative pathogens even at concentrations of 64 μg/ml (FIG. 10). By contrast, linezolid conjugated with 3FG6P (Lzd-3FG6P) successfully inhibited the growth of both Gram positive and Gram-negative pathogens at a concentration range of 2-8 μg/ml (FIG. 10 and Table 2 below). These results clearly demonstrate that antimicrobial activity of linezolid was expanded to gram-negative pathogens by conjugating with 3FG6P.

TABLE 2
Minimal inhibitory concentration of linezolid
and linezolid conjugated with 3FG6P

| | MIC (μg/ml) | |
|---|---|---|
| | | Linezolid- |
| | Linezolid | 3FG6P |
| Staphylococcus aureus ATCC 25923 | <2 | <2 |
| Klebsiella pneumoniae ATCC 35657 | >64 | <2 |
| Acinetobacter baumannii ATCC BAA1605 | >64 | <2 |
| Escherichia coli ATCC 25922 | >64 | 4 |
| Enterobacter cloacae ATCC13047 | >64 | <2 |
| Enterobacter aerogenes ATCC13048 | >64 | 4 |
| Salmonella typhimurium ATCC 14028 | >64 | 8 |

### Example 5: In Vivo Antimicrobial Activity of Linezolid and Linezolid Conjugated with 3FG6P

To test the in vivo antimicrobial activity of linezolid and linezolid conjugated with 3FG6P, clinical E. coli strains were constructed that constitutively express a bioluminescent light signal using pLuxABCDE plasmid. See Karsi A. Lawrence M L. Broad host range fluorescence and bioluminescence expression vectors for Gram-negative bacteria. Plasmid. 2007; 57(3):286-95. Epub 2007/01/09. doi: 10.1016/j.plasmid.2006.11.002. PubMed PMID: 17207855 for real time monitoring of the progress of infections. Six- to eight-week old female C57BL/6 mice were purchased from Harlan laboratory and were housed and maintained according to the protocol approved by the institutional animal care and use committee at Mississippi State University. Animals (n=6/group) were transurethrally infected with 50 μl of bioluminescent E. coli strain suspended in PBS (2×10⁹ CFU/ml). Animals were treated with an intraperitoneal injection of linezolid (80 mg/kg), linezolid conjugated with 3FG6P (80 mg/kg) or PBS control at 2, 24, and 48 hours after infection. The progress of the infection was monitored by measuring the bioluminescent light signal using an IVIS Lumina X R small animal imaging system. After 72 hours infection, animals were humanely euthanized and the bladder and kidney samples were collected, homogenized, and serially diluted, and the serial dilutions were plated on blood agar to determine the bacterial burden. A transurethral inoculation of E. coli established persistent infections in the kidney and bladder in the absence of antibiotic treatment (PBS control group). The mean bacterial counts in the kidney and bladder were log₁₀ 6.328±0.132 and log₁₀ 6.171+0.155 CFU/g, respectively (FIG. 12). Treatment with linezolid did not reduce bacterial counts in the kidney and bladder, while treatment with linezolid conjugated with 3FG6P (Lzd-3FG6P) completely cleared the infection from the kidney and nearly completely cleared the infection from the bladder. These results clearly demonstrate that conjugation of 3FG6P makes linezolid highly effective for control of urinary tract infections caused by E. coli.

### Example 6: Improving Antimicrobial Activity of Fosfomycin by Co-Administration with 4FG6P or 3FG6P

Fosfomycin is a bactericidal antibiotic with broad spectrum activity against both gram positive and gram negative bacteria since it is transported through the glycerol-3-phosphate transporter (GlpT) system and the glucose-6-phosphate transporter (UhpT) system, which systems are highly conserved in most bacteria. See Sastry S, Doi Y. 2016. Fosfomycin: Resurgence of an old companion. J Infect Chemother 22:273-80; Kahan F M, Kahan J S, Cassidy P J, Kropp H. 1974. The mechanism of action of fosfomycin (phosphonomycin). Ann N Y Acad Sci 235:364-86; Park J Y, Kim J W, Moon B Y, Lee J, Fortin Y J, Austin F W, Yang S J, Seo K S. 2015. Characterization of a novel two-component regulatory system, HptRS, the regulator for the hexose phosphate transport system in Staphylococcus aureus. Infect Immun 83:1620-8; and Sit B, Crowley S M, Bhullar K, Lai C C, Tang C, Hooda Y, Calmettes C, Khambati H, Ma C, Brumell J H, Schryvers A B, Vallance B A, Moraes T F. 2015. Active Transport of Phosphorylated Carbohydrates Promotes Intestinal Colonization and Transmission of a Bacterial Pathogen. PLoS Pathog 11:e1005107.

Fosfomycin is not metabolized in the liver, and is primarily excreted unchanged in the urine by glomerular filtration. See Segre G, Bianchi E, Cataldi A, Zannini G. 1987. Pharmacokinetic profile of fosfomycin trometamol (Monuril). Eur Urol 13 Suppl 1:56-63. Therefore, it is approved by the FDA for oral administration to treat uncomplicated urinary tract infections (UTIs). Fosfomycin has low toxicity and good distribution in serum, kidneys, the bladder wall, lungs, inflamed tissues, bone, cerebrospinal fluid, abscess fluid, and heart valves. See Schintler M V, Traunmuller F, Metzler J, Kreuzwirt G, Spendel S, Mauric O, Popovic M, Scharnagl E, Joukhadar C. 2009. High fosfomycin concentrations in bone and peripheral soft tissue in diabetic patients presenting with bacterial foot infection. J Antimicrob Chemother 64:574-8. Fosfomycin inhibits the first step of peptidoglycan synthesis by blocking the MurA enzyme, catalyzing synthesis of early peptidoglycan precursors (6). This unique mechanism of action confers the synergistic effect of fosfomycin against ESAPKE pathogens in combination with other antibiotics such as beta-lactams, aminoglycosides, and fluoroquinolones. See Sastry S, Doi Y. 2016. Fosfomycin: Resurgence of an old companion. J Infect Chemother 22:273-80.

11. Falagas M E, Kastoris A C, Karageorgopoulos D E, Rafailidis P I. 2009. Fosfomycin for the treatment of infections caused by multidrug-resistant non-fermenting Gram-negative bacilli: a systematic review of microbiological, animal and clinical studies. Int J Antimicrob Agents 34:111-20; Walsh C C, Landersdorfer C B, McIntosh M P, Peleg A Y, Hirsch E B, Kirkpatrick C M, Bergen P J. 2016. Clinically relevant concentrations of fosfomycin combined with polymyxin B, tobramycin or ciprofloxacin enhance bacterial killing of Pseudomonas aeruginosa, but do not suppress the emergence of fosfomycin resistance. J Antimicrob Chemother 71:2218-29; and Ferrara A, Dos Santos C, Cimbro M, Gialdroni Grassi G. 1997. Effect of different combinations of sparfloxacin, oxacillin, and fosfomycin against methicillin-resistant staphylococci. Eur J Clin Microbiol Infect Dis 16:535-7. These characteristics make fosfomycin an important therapeutic option against MDR ESAPKE pathogens. Thus, there is an increasing interest in exploring the extended use of fosfomycin to treat other indications caused by MDR pathogens (Ref-KSS). However, oral administration of fosfomycin showed a low oral bioavailability of 30-37% and lower distribution to other tissues than in the bladder. Furthermore, the minimum inhibitory concentration (MIC) breakpoint of fosfomycin is relatively higher (8-32 mg/liter for Enterobacteriaceae) than other antibiotics and fosfomycin-resistant strains producing fosfomycin-modifying enzymes (FosA, FosB, and FosX) could be selected and rapidly spread.

To test whether 3FG6P and 4FG6P can potentiate the efficacy of fosfomycin, clinical fosfomycin resistant UTI E. coli isolates harboring the fosA gene and S. aureus COL strain were obtained. Overnight cultures of these bacteria were grown in brain heart infusion (BHI) broth diluted to 0.5 McFarland turbidity and inoculated into fresh BHI broth supplemented with various concentrations of fosfomycin alone or fosfomycin supplemented with 3FG6P for E. coli or 4FG6P for S. aureus (50 μM).

As shown in FIGS. 13A to 13D, both fosfomycin resistant E. coli and S. aureus allowed growth in BHI supplemented up to 128 μg/ml of fosfomycin alone. In contrast, the growth of both bacteria in BHI supplemented with 50 μM of 3FG6P or 4FG6P was considerably inhibited at low concentrations of fosfomycin. Particularly, with 50 μM of 3FG6P, the growth of E. coli was completely inhibited even at 2 μg/ml of fosfomycin. Similarly, the growth of fosfomycin resistant S. aureus was completely inhibited at 32 μg/ml of fosfomycin and significantly delayed at lower concentrations.

These results clearly demonstrated that induction of UhpT expression by activating the three-component regulatory system significantly enhanced the fosfomycin efficacy to a level sufficient to reverse the resistance mechanism by fosfomycin modifying enzymes.

### Example 7: In Vivo Antimicrobial Activity of Fosfomycin and Fosfomycin Co-Administered with 3FG6P

To test the in vivo antimicrobial activity of fosfomycin and fosfomycin co-administered with 4FG6P, S. aureus COL strain constitutively expressing a bioluminescent light signal using pLuxABCDE plasmid were generated for real time monitoring of the progress of infections. Six to eight-week old female C57BL/6 mice were purchased from Harlan laboratory and were housed and maintained according to the protocol approved by the institutional animal care and use committee at Mississippi State University.

Animals (n=3/group) were intraperitoneally infected with 50 μl of bioluminescent S. aureus strain suspended in PBS (2×10⁹ CFU/ml). Animals were treated with an intraperitoneal injection of fosfomycin (3 mg/kg) alone or fosfomycin co-administered with 4FG6P (50 μg/kg) or PBS control at 2 and 24 hours after infection. The progress of the infection was monitored by measuring the bioluminescent light signal using an IVIS Lumina XR small animal imaging system.

After 48 hours infection, animals were humanely euthanized and the bacterial burdens in the lung, kidney, liver, spleen, and peritoneal lavage were determined. Animals treated with PBS or fosfomycin alone showed mean bacterial counts ranging from 5.34±0.154 to 6.47±0.115 CFU/g, respectively. By contrast, a treatment of fosfomycin co-administered with 4FG6P completely cleared infections at peritoneal lavage, lung, and kidney and significantly reduced the bacterial burdens more than 4 log scales at liver and spleen (FIG. 14).

1. A conjugated drug comprising a drug conjugated to 1DG6P of formula (D1): 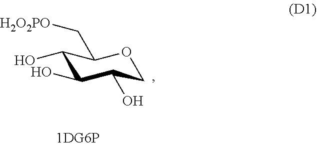
or a fluorinated hexose phosphate.

2. The conjugated drug of claim 1, wherein the drug is an antibiotic.

3. The conjugated drug of claim 2, wherein the drug is selected from the group consisting of linezolid, Fosfomycin, streptozotocin, floxuridine, a nitrofuranylamide of the formula: 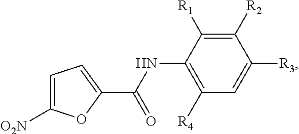
wherein R₁=H, R₂=H, R₃=H and R₄=H; a quinazolindiamine of the formula: 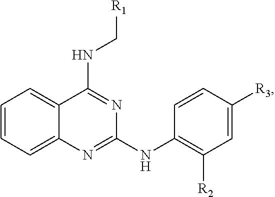
wherein R₁=2-(tetrahydrofuryl), R₂=OCH₃ and R₃=H; a nitrofuanylethenyl of the formula: 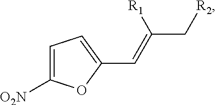
wherein R₁=benzimidazole-2-yl and R₂=CN; a benzylphenylethylamine of the formula: 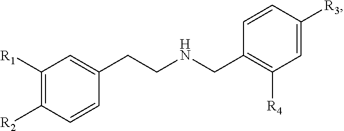
and
wherein R₁=OCH₃, R₂=OCH₃ and R₃=F, R₄=H; and a salicylanilide of the formula: 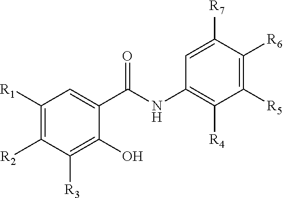
wherein R₁-R₃=Br, R₄=H and R₅-R₇=Cl.

4. The conjugated drug of claim 2, wherein the drug is selected from linezolid and fosfomycin.

5. The conjugated drug of claim 1, wherein the drug is conjugated to the fluorinated hexose phosphate selected from the group consisting of 3-fluoro-glucose-6-phosphate, 4-fluoro-glucose-6-phosphate, 3-deoxy-3,3-difluoroglucose, 2-deoxy-2-fluoroglucose, the 2,2-difluorinated derivative of 2-deoxy-2-fluoroglucose of formula (B2): 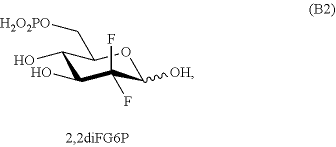
2,3-dideoxy-2,3-difluoroglucose, 2,3-dideoxy-2,2,3,3-tetrafluorinated analog of formula (C2): 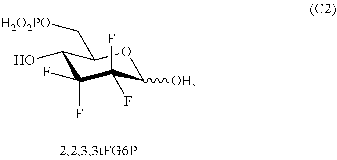
a 3-fluorinated analog of 1DG6P of formula (D2): 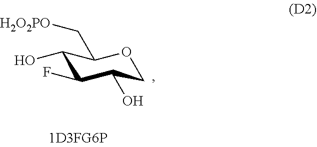
and
4-deoxy-4-fluoroglucose, and phosphate analogs of formulae (F1) and F2): 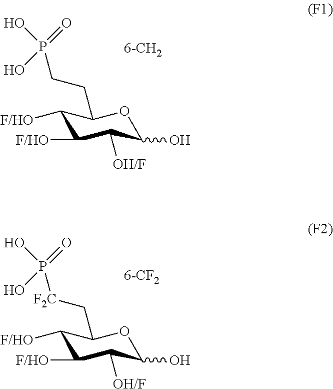
wherein F/OH indicates that the substituent can be either —F or —OH, subject to the proviso that each compound of these formulae must have at least one substituent that is —F.

6. The conjugated drug of claim 1, wherein the drug is conjugated to 3-fluoro-glucose-6-phosphate.

7. The conjugated drug of claim 1, wherein the drug is conjugated to 4-fluoro-glucose-6-phosphate.

8. A method of enhancing UhpT uptake of a drug, as compared to uptake of an unconjugated form of the drug, comprising a step of conjugating a non-metabolizable hexose phosphate that constitutively activates a HptARS regulatory system and induces expression of hexose phosphate transporter (UhpT), to the drug.

9. A method of conjugating a non-metabolizable hexose phosphate to a drug to enhance UhpT uptake of the conjugated drug, as compared to uptake of an unconjugated form of the drug, said method comprising a step of reacting the drug with the non-metabolizable hexose phosphate of claim 1.

10. The method of claim 8, wherein the drug is an antibiotic.

11. The method of claim 10, wherein the antibiotic is selected from linezolid and fosfomycin.

12. The method of claim 8, wherein the non-metabolizable hexose phosphate is 3-fluoro-glucose-6-phenylated phosphate.

13. The method of claim 8, wherein the non-metabolizable hexose phosphate is 4-fluoro-glucose-6-phenylated phosphate.

14. A method to conjugate a 3-fluoro-glucose-6-phosphate moiety with a linezolid moiety comprising steps of:
reacting (S)—N-[[3-[3-fluoro-4-(N-1-piperazinyl)phenyl]-2-oxo-5-oxazolidinyl]methyl]acetamide of formula (11): 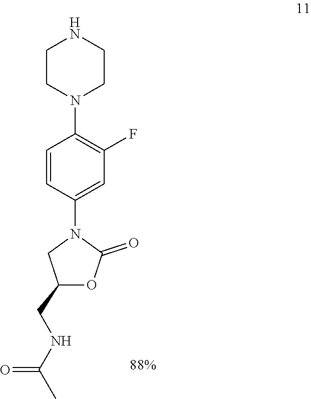 [SMILES: CC(=O)NC[C@H]1CN(c2ccc(N3CCNCC3)c(F)c2)C(=O)O1]
with γ-butyrolactone to provide an amide of the formula (14): 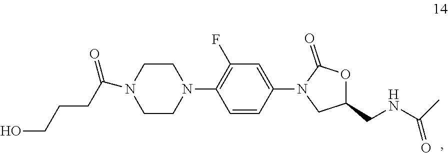 [SMILES: CC(=O)NC[C@H]1CN(c2ccc(N3CCN(C(=O)CCCO)CC3)c(F)c2)C(=O)O1]
and
coupling the amide of the formula (14) with a 3-fluoro-glucose-6-phenylated phosphate of formula (13): 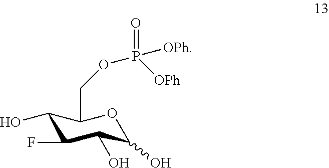
by an acid-catalyzed glycosylation reaction to produce a product of the formula (15): 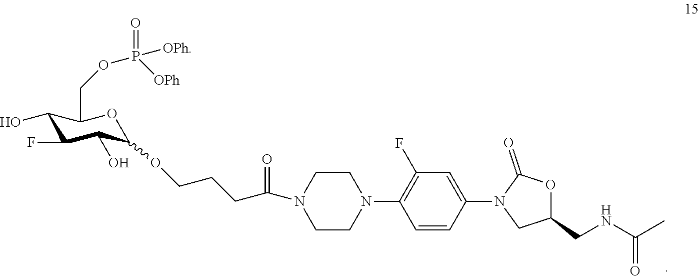
and
deprotection of the product of the formula (15) to provide a conjugated product of the formula (16):

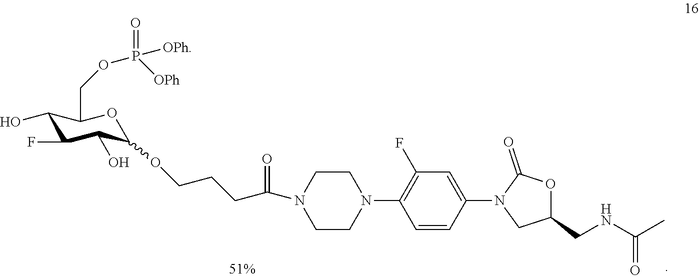

15. A method of treating a bacterial infection comprising administering to a patient with said bacterial infection a composition comprising the conjugated antibiotic of claim 2.

16. (canceled)

17. A method of treating a bacterial infection comprising a step of co-administering one or more antibiotics with at least one non-metabolizable hexose phosphate or fluorinated hexose phosphate.

18. The method of claim 17, wherein the hexose phosphate or the fluorinated hexose phosphate is selected from the group consisting of 3-fluoro-glucose-6-phosphate, 4-fluoro-glucose-6-phosphate, 3-deoxy-3,3-difluoroglucose, 2-deoxy-2-fluoroglucose, the 2,2-difluorinated derivative of 2-deoxy-2-fluoroglucose of formula (B2): 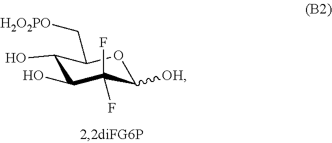
2,3-dideoxy-2,3-difluoroglucose, 2,3-dideoxy-2,2,3,3-tetrafluorinated analog of formula (C2): 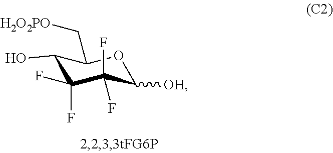
1DG6P of formula (D1): 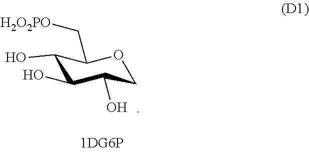
a corresponding 3-fluorinated analog of formula (D2): 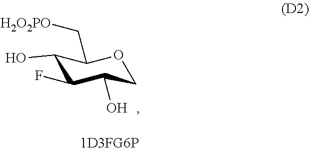
and
4-deoxy-4-fluoroglucose, and phosphate analogs of formulae (F1) and F2): 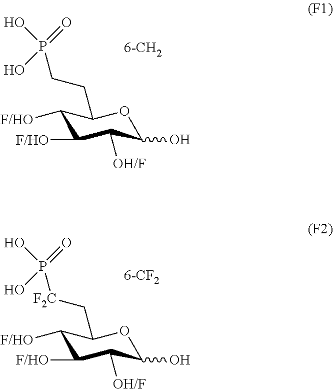
wherein F/OH indicates that the substituent can be either —F or —OH, subject to the proviso that each compound of these formulae must have at least one substituent that is —F.

19-20. (canceled)

21. The conjugated drug of claim 3, wherein the drug is conjugated to the fluorinated hexose phosphate selected from the group consisting of 3-fluoro-glucose-6-phosphate, 4-fluoro-glucose-6-phosphate, 3-deoxy-3,3-difluoroglucose, 2-deoxy-2-fluoroglucose, the 2,2-difluorinated derivative of 2-deoxy-2-fluoroglucose of formula (B2): 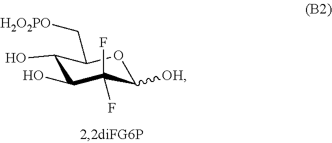
2,3-dideoxy-2,3-difluoroglucose 2,3-dideoxy-2,2,3,3-tetrafluorinated analog of formula (C2): 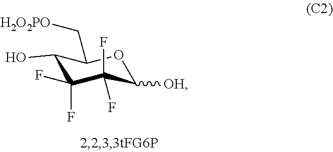
a 3-fluorinated analog of 1DG6P of formula (D2): 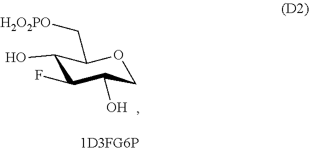
and
4-deoxy-4-fluoroglucose, and phosphate analogs of formulae (F1) and F2): 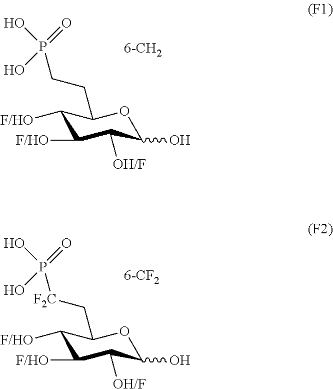
wherein F/OH indicates that the substituent can be either —F or —OH, subject to the proviso that each compound of these formulae must have at least one substituent that is —F.

22. The conjugated drug of claim 3, wherein the drug is conjugated to 3-fluoro-glucose-6-phosphate.

23. The conjugated drug of claim 3, wherein the drug is conjugated to 4-fluoro-glucose-6-phosphate.
